(19) United States
(12) Patent Application Publication
(10) Pub. No.: US 2008/0124455 A1
(43) Pub. Date: May 29, 2008

(54) ORGANIC LIGHT EMITTING COMPOUND, ORGANIC LIGHT EMITTING DEVICE COMPRISING THE SAME, AND METHOD OF MANUFACTURING THE ORGANIC LIGHT EMITTING DEVICE

(75) Inventors: Dong-woo SHIN, Seoul (KR); Eun-sil HAN, Yongin-si (KR); Woon-jung PAEK, Yongin-si (KR); Yi-yeol LYU, Yongin-si (KR); O-hyun KWON, Seoul (KR); Myeong-suk KIM, Suwon-si (KR); Byoung-ki CHOI, Hwaseong-si (KR); Jhun-mo SON, Yongin-si (KR); Young-mok SON, Hwaseong-si (KR)

Correspondence Address: CANTOR COLBURN, LLP, 20 Church Street, 22nd Floor, Hartford, CT, 06103, US

(73) Assignee: SAMSUNG ELECTRONICS CO., LTD., Suwon-si (KR)

(21) Appl. No.: 11/767,316

(22) Filed: Jun. 22, 2007

(30) Foreign Application Priority Data
Nov. 24, 2006 (KR) 10-2006-0117250

(51) Int. Cl. B05D 5/12; C07D 209/56; C07D 209/80; C07D 487/22; H01L 51/50

(52) U.S. Cl. 427/66; 313/504; 548/417; 548/418; 548/420

(57) ABSTRACT

Provided are an organic light emitting compound represented by Formula 1 below, an organic light emitting device comprising the same, and a method of manufacturing the organic light emitting device:

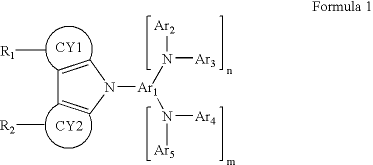

where CY1 and CY2 are each independently a fused C₆-C₅₀ aromatic ring, Ar₁ is a substituted or unsubstituted C₆-C₅₀ arylene group, Ar₂, Ar₃, Ar₄, and Ar₅ are each independently a substituted or unsubstituted C₆-C₅₀ aryl group, m and n are independently 0-3, and R₁ and R₂ are substituent groups. An organic light emitting device comprising the organic light emitting compound has low turn-on voltage, high efficiency, high color purity and high luminance.

This application claims priority to Korean Patent Application No. 10-2006-0117250, filed on Nov. 24, 2006, and all the benefits accruing therefrom under 35 U.S.C. §119(a), the disclosure of which is incorporated herein by reference in its entirety.

### BACKGROUND OF THE INVENTION

1. Field of the Invention

The present invention relates to an organic light emitting compound, an organic light emitting device comprising the same, and a method of manufacturing the organic light emitting device, and more particularly, to an organic light emitting compound that provides good electrical properties, high thermal stability and high photochemical stability, and that provides low turn-on voltage, high color purity and high luminance when the organic light emitting compound is used in an organic light emitting device, an organic light emitting device comprising the same, and a method of manufacturing the organic light emitting device.

2. Description of the Related Art

Light emitting devices, which are self-light emitting devices, typically have wide viewing angles, excellent contrast, and quick response time. Light emitting devices can be categorized into inorganic light emitting devices and organic light emitting devices (“OLED”) according to the materials used to form the emission layer of the light emitting device. Organic light emitting devices are brighter, and have a lower operating voltage and quicker response time when compared to inorganic light emitting devices, and can exhibit multi color images.

In general, an organic light emitting device has an anode/organic emission layer/cathode layered structure. An organic light emitting device can also have various other structures with other layers included in the stacked layers, such as an anode/hole injection layer/hole transport layer/emission layer/electron transport layer/electron injection layer/cathode structure, or an anode/hole injection layer/hole transport layer/emission layer/hole blocking layer/electron transport layer/electron injection layer/cathode structure. Materials used in organic light emitting devices can be classified into vacuum deposition materials and solution coating materials depending on the method of preparing an organic layer of the organic light emitting devices. The vacuum deposition material can have a vapor pressure of greater than or equal to 10⁻⁶ torr or more at a temperature of 500° C. or less, and may be a low molecular material having a molecular weight of 1,200 g/mol or less. The solution coating material should have high solubility in an appropriate solvent so that it can be prepared as a solution, and suitable materials can include primarily an aromatic group or a heterocyclic ring.

When organic light emitting devices are manufactured using vacuum deposition, the manufacturing cost increases because of the use of a vacuum system. In addition, when a shadow mask method is used for preparing pixels for natural color display, it is difficult to prepare high resolution pixels. On the other hand, when organic light emitting devices are manufactured using a solution coating method such as for example inkjet printing, screen printing or spin coating, the manufacturing method can be simple and inexpensive, and the organic light emitting devices can have relatively good resolution when compared with organic light emitting devices manufactured using a shadow mask method.

However, for OLED's that include blue light-emitting organic molecules, a material that can be used in solution coating is inferior to a material that can be used in vacuum deposition in terms of thermal stability, color purity and the like. In addition, although such materials having good performance are used for preparing an organic layer, problems can arise where the material gradually crystallizes after the organic layer is prepared such that the size (largest dimension or length) of the crystal eventually formed approximates that of a wavelength of visible light. As a result, visible light diffuses so that a whitening phenomenon can occur, and pin holes or the like can form, thereby causing degradation of the device. Japanese Patent Publication No. 1999-003782 discloses anthracene substituted with two naphthyl groups as a compound that can be used in an emission layer or a hole injection layer. However, the compound does not have sufficiently good solubility with respect to any useful solvent, and organic light emitting devices using such compounds therefore do not have satisfactory characteristics.

Accordingly, there is demand for a compound that can form an organic layer having excellent properties that can be used in an organic light emitting device. In addition, there is need for development of an organic light emitting device that has improved turn-on voltage, high luminance, high efficiency and high color purity using a blue emitting compound that has high thermal stability and can form an organic layer having excellent properties.

### BRIEF SUMMARY OF THE INVENTION

In consideration of the deficiencies of the prior art, in an embodiment, an organic light emitting compound having good solubility and high thermal stability is provided.

In another embodiment, an organic light emitting device includes the organic light emitting compound, and has improved turn-on voltage, efficiency, color purity and luminance.

In another embodiment, a method of manufacturing an organic light emitting device using the organic light emitting compound.

In an embodiment, an organic light emitting compound can be represented by Formula 1 below:

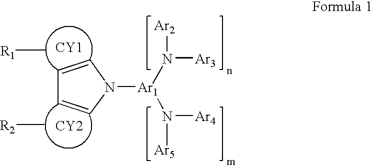

where CY1 and CY2 are each independently a fused C₆-C₅₀ aromatic ring;

Ar₁ is a substituted or unsubstituted C₆-C₅₀ arylene group;

Ar₂, Ar₃, Ar₄ and Ar₅ are each independently a substituted or unsubstituted C₆-C₅₀ aryl group, Ar₂ and Ar₃ are separate or bound to each other to form a substituted or unsubstituted C₁₃-C₁₀₀ heteroaryl group containing N, and Ar₄ and Ar₅ are separate or bound to each other to form a substituted or unsubstituted C₁₃-C₁₀₀ heteroaryl group containing N;

R₁ and R₂ represent one or more substituent groups and are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; n and m are each independently an integer of 0 through 3; and

wherein Ar₁ is a substituted or unsubstituted C₆-C₅₀ aryl group when n and m are both 0, and CY1 and CY2 are unsubstituted where R₁ and R₂ are respectively each a hydrogen.

In a specific embodiment, there is provided an organic light emitting compound represented by Formula 2 below:

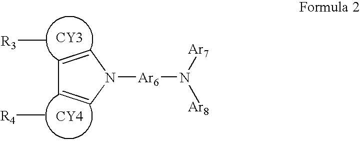

where CY3 and CY4 are each independently a fused benzene ring or a fused naphthalene ring;

Ar₆ is a substituted or unsubstituted C₆-C₅₀ arylene group;

Ar₇ and Ar₈ are each independently a substituted or unsubstituted C₆-C₅₀ aryl group or a substituted or unsubstituted C₁₃-C₁₀₀ heteroaryl group; R₁ and R₂ represent one or more substituent groups and are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; and

wherein CY3 and CY4 are not simultaneously fused benzene rings, and CY3 and CY4 are unsubstituted where R₃ and R₄ are respectively each a hydrogen.

In another embodiment, there is provided an organic light emitting device comprising: a first electrode; a second electrode; and an organic layer interposed between the first electrode and the second electrode, wherein the organic layer comprises the organic light emitting compound described above.

### BRIEF DESCRIPTION OF THE DRAWINGS

The above and other features and advantages of the present invention will become more apparent by describing in detail exemplary embodiments thereof with reference to the attached drawings in which:

FIGS. 1A through 1C are schematic cross-sectional views illustrating structures of exemplary organic light emitting devices according to embodiments;

FIG. 2 is a graph illustrating UV and photoluminescence (“PL”) spectra of a solution comprising an exemplary compound (Compound 5) according to an embodiment; and

FIG. 3 is a graph showing efficiency of an exemplary organic light emitting device (Sample 2) manufactured using Compound 5, according to an embodiment.

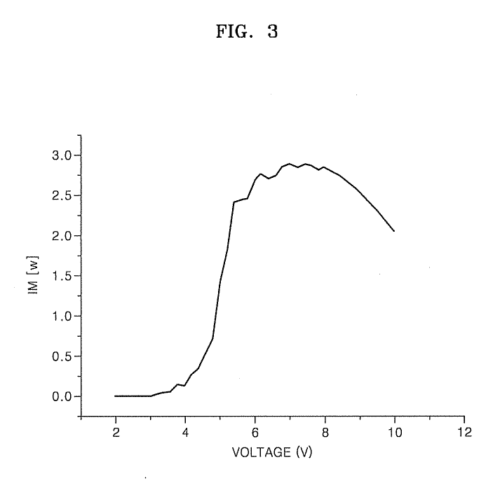

### DETAILED DESCRIPTION OF THE INVENTION

Hereinafter, the present invention will be described more fully with reference to the accompanying drawings, in which exemplary embodiments of the invention are shown.

It will be understood that when an element is referred to as being “on” another element, it can be directly on the other element or intervening elements may be present therebetween. In contrast, when an element is referred to as being “disposed on”, “interposed between”, or “formed on” another element, the elements are understood to be in at least partial contact with each other, unless otherwise specified.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

An organic light emitting compound according to an embodiment is represented by Formula 1 below:

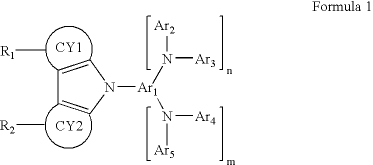

where CY1 and CY2 are each independently a fused C₆-C₅₀ aromatic ring;

Ar₁ is a substituted or unsubstituted C₆-C₅₀ arylene group;

Ar₂, Ar₃, Ar₄ and Ar₅ are each independently a substituted or unsubstituted C₆-C₅₀ aryl group, Ar₂ and Ar₃ are separate (where separate, as used herein in this context, means discrete Ar groups covalently bonded to a common heteroatom but not also covalently bonded to one another) or bound to each other to form a substituted or unsubstituted C₁₃-C₁₀₀ heteroaryl group containing N, and Ar₄ and Ar₅ are separate or bound to each other to form a substituted or unsubstituted C₁₃-C₁₀₀ heteroaryl group containing N;

R₁ and R₂ represent one or more substituent groups and are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; n and m are each independently an integer of 0 through 3; and

wherein Ar₁ is a substituted or unsubstituted C₆-C₅₀ aryl group when n and m are both 0, and CY1 and CY2 are unsubstituted where R₁ and R₂ are each respectively a hydrogen.

The aryl group is a monovalent group having an aromatic ring system, and can include at least two ring systems. The at least two ring systems may be attached or fused together. The heteroaryl group is a group in which at least one carbon atom of the aryl group is substituted with at least one selected from the group consisting of N, O, S and P. Meanwhile, the cycloalkyl group refers to an alkyl group having a ring system, and the heterocycloalkyl group is a group in which at least one carbon atom of the cycloalkyl group is substituted with at least one selected from the group consisting of N, O, S and P.

In another embodiment, an organic light emitting compound is represented by Formula 2 below:

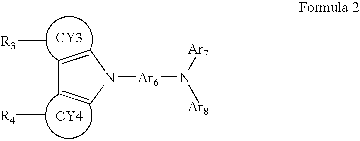

where CY3 and CY4 are each independently a fused benzene ring or a fused naphthalene ring;

Ar₆ is a substituted or unsubstituted C₅-C₅₀ arylene group;

Ar₇ and Ar₈ are each independently a substituted or unsubstituted C₆-C₅₀ aryl group or a substituted or unsubstituted C₁₃-C₁₀₀ heteroaryl group; R₃ and R₄ represent one or more substituent groups and are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; and

wherein CY3 and CY4 are not simultaneously fused benzene rings, and CY3 and CY4 are unsubstituted where R₃ and R₄ are respectively each a hydrogen.

Of Formulas 1 or 2, carbazole derivatives and the aryl group bound thereto increase thermal stability and photochemical stability of the organic light emitting compound represented by Formula 1 or 2. In addition, R₁ through R₄, which are substituents, increase solubility and an amorphous property of the organic light emitting compound generally represented by Formula 1 so as to improve film processability. The organic light emitting compound represented by Formula 1 or 2 is a material suitable for forming an organic layer of an organic light emitting device, wherein the organic layer is interposed between a first electrode and a second electrode. The organic light emitting compound represented by Formula 1 is suitable for an organic layer of an organic light emitting device, in particular, an emission layer, a hole injection layer or a hole transport layer, and can also be used as a dopant material in addition to a host material.

Substituents of the alkyl group, the alkoxy group, the arylene group, the aryl group, the heteroaryl group, the cycloalkyl group and the heterocycloalkyl group may be at least one selected from the group consisting of —F; —Cl; —Br; —CN; —NO₂; —OH; an unsubstituted C₁-C₅₀ alkyl group or a C₁-C₅₀ alkyl group substituted with —F, —Cl, —Br, —CN, —NO₂ or —OH; an unsubstituted C₁-C₅₀ alkoxy group or a C₁-C₅₀ alkoxy group substituted with —F, —Cl, —Br, —CN, —NO₂ or —OH; an unsubstituted C₆-C₅₀ aryl group or a C₆-C₅₀ aryl group substituted with a C₁-C₅₀ alkyl group, a C₁-C₅₀ alkoxy group, —F, —Cl, —Br, —CN, —NO₂ or —OH; an unsubstituted C₂-C₅₀ heteroaryl group or a C₂-C₅₀ heteroaryl group substituted with a C₁-C₅₀ alkyl group, a C₁-C₅₀ alkoxy group, —F, —Cl, —Br, —CN, —NO₂ or —OH; an unsubstituted C₅-C₅₀ cycloalkyl group or a C₅-C₅₀ cycloalkyl group substituted with a C₁-C₅₀ alkyl group, a C₁-C₅₀ alkoxy group, —F, —Cl, —Br, —CN, —NO₂ or —OH; an unsubstituted C₅-C₅₀ heterocycloalkyl group or a C₅-C₅₀ heterocycloalkyl group substituted with a C₁-C₅₀ alkyl group, a C₁-C₅₀ alkoxy group, —F, —Cl, —Br, —CN, —NO₂ or —OH; and —N(Z₉)(Z₁₀) where Z₉ and Z₁₀ may be each independently a hydrogen atom, a C₁-C₅₀ alkyl group, or a C₆-C₅₀ aryl group substituted with a C₁-C₅₀ alkyl group.

In a specific embodiment, CY1 can be selected from the group consisting of a benzene ring, a toluene ring, a pentalene ring, an indene ring, a naphthalene ring, a biphenylene ring, an anthracene ring, an azulene ring, a heptalene ring, an acenaphthylene ring, a phenalene ring, a fluorene ring, a tetracene ring, a triphenylene ring, a pyrene ring, a chrysene ring, an ethyl-chrysene ring, a phycene ring, a perylene ring, a pentaphene ring, a pentacene ring, a tetraphenylene ring, a hexaphene ring, a hexacene ring, a rubicene ring, a coronene ring, a trinaphthylene ring, a heptaphene ring, a heptacene ring, a pyranthrene ring, an ovalene ring, a fluoranthrene ring, a benzofluoranthrene ring and derivatives thereof; and

CY2 can be selected from the group consisting of a pentalene ring, an indene ring, an anthracene ring, an azulene ring, a heptalene ring, an acenaphthylene ring, a phenalene ring, a fluorene ring, a tetracene ring, a triphenylene ring, a pyrene ring, a chrysene ring, an ethyl-chrysene ring, a phycene ring, a perylene ring, a pentaphene ring, a pentacene ring, a tetraphenylene ring, a hexaphene ring, a hexacene ring, a rubicene ring, a coronene ring, a trinaphthylene ring, a heptaphene ring, a heptacene ring, a pyranthrene ring, an ovalene ring, a fluoranthrene ring, a benzofluoranthrene ring and derivatives thereof.

The term “derivatives” used in the present application refers to a group in which at least one hydrogen atom of the groups described above is substituted with the substituents described above.

More particularly, Ar₂, Ar₃, Ar₄, Ar₅, Ar₇ and Ar₈ may be each independently selected from the group consisting of a phenyl group, a tolyl group, a biphenyl group, a pentarenyl group, an indenyl group, a naphthyl group, a biphenylenyl group, an anthracenyl group, an azulenyl group, a heptalenyl group, an acenaphthylenyl group, a phenarenyl group, a fluolenyl group, a methylanthryl group, a phenanthrenyl group, a triphenylene group, a pyrenyl group, a chrysenyl group, an ethyl-chrysenyl group, a pycenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovarenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolynyl group, a oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a di(C₆-C₅₀ aryl)aminophenyl group and derivatives thereof. Of those groups, Ar₂, Ar₃, Ar₄, Ar₅, Ar₇ and Ar₈ may be each independently selected from the group consisting of a phenyl group, a tolyl group, a naphthyl group, an anthracenyl group, a pyrenyl group, a phenanthrenyl group, a fluorenyl group, an imidazolynyl group, an indolyl group, a quinolinyl group, a 2,3-di-p-tolylaminophenyl group and a naphtho[2,3-c]carbazolyl group.

More particularly, Ar₁ and Ar₆ may be each independently selected from the group consisting of a phenylene group, a biphenylene group, a p-terphenylene group, a 1,3,5-triphenylbenzylene group, a tolylene group, a biphenylene group, a pentalenylene group, an indenylene group, a naphthylene group, a biphenylenylene group, an anthracenylene group, an azulenylene group, a heptalenylene group, an acenaphthylenylene group, a phenalenylene group, a fluolenylene group, a methylanthrylene group, a phenanthrenylene group, a triphenylenylene group, a pyrenylene group, a chrysenylene group, an ethyl-chrysenylene group, a picenylene group, a perylenylene group, a chloroperylenylene group, a pentaphenylene group, a pentacenylene group, a tetraphenylenylene group, a hexaphenylene group, a hexacenylene group, a rubicenylene group, a coronenylene group, a trinaphthylenylene group, a heptaphenylene group, a heptacenylene group, a fluorenylene group, a pyranthrenylene group, an ovalenylene group, a carbazolylene group, a thiophenylene group, an indolylene group, a purinylene group, a benzimidazolylene group, a quinolinylene group, a benzothiophenylene group, a parathiazinylene group, a pyrrolylene group, a pyrazolylene group, an imidazolylene group, an imidazolinylene group, a oxazolylene group, a thiazolylene group, a triazolylene group, a tetrazolylene group, an oxadiazolylene group, a pyridinylene group, a pyridazinylene group, a pyrimidinylene group, a pyrazinylene group, a thianthrenylene group, a di(C₆-C₅₀ aryl)aminophenylene group and derivatives thereof.

Of those groups described above, Ar₁ and Ar₆ may be each independently a phenylene group, a biphenylene group, a p-terphenylene group, a 1,3,5-triphenylbenzylene group, a naphthylene group, an anthracenylene group, a pyrenylene group, a phenanthrenylene group, a fluolenylene group, an imidazolynylene group, an indolylene group, a quinolinylene group and a 2,3-di-p-tolylaminophenylene group.

Herein, when n and m are both 0, specific arylene groups of Ar₁ described above are replaced as specific aryl groups each corresponding thereto.

More particularly, R₁ through R₄ may be each independently selected from the group consisting of a hydrogen atom, a C₁-C₅₀ alkyl group, a C₁-C₅₀ alkoxy group, a phenyl group, a tolyl group, a biphenyl group, a pentarenyl group, an indenyl group, a naphthyl group, a biphenylenyl group, an anthracenyl group, an azulenyl group, a heptalenyl group, an acenaphthylenyl group, a phenarenyl group, a fluolenyl group, a methylanthryl group, a phenanthrenyl group, a triphenylenyl group, a pyrenyl group, a chrysenyl group, an ethyl-chrysenyl group, a pycenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovarenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolynyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolidinyl group, a piperidinyl group, a piperazinyl group, a morpholinyl group, a di(C₆-C₅₀ aryl)amino group, a di(C₆-C₅₀ aryl)aminophenyl group, a tri(C₆-C₅₀ aryl)silyl group and derivatives thereof.

Of those groups described above, R₁ through R₄ may be each independently methyl, methoxy, a phenyl group, a tolyl group, a naphthyl group, a pyrenyl group, a phenanthrenyl group, a fluorenyl group, an imidazolynyl group, an indolyl group, a quinolinyl group, a diphenylamino group, a 2,3-di-p-tolylaminophenyl group, a naphthylphenylamino group, a dinaphthylamino group and a triphenylsilyl group.

More particularly, organic light emitting compounds according to other embodiments can be compounds represented by, but not limited to, Formulae 3 through 18 below:

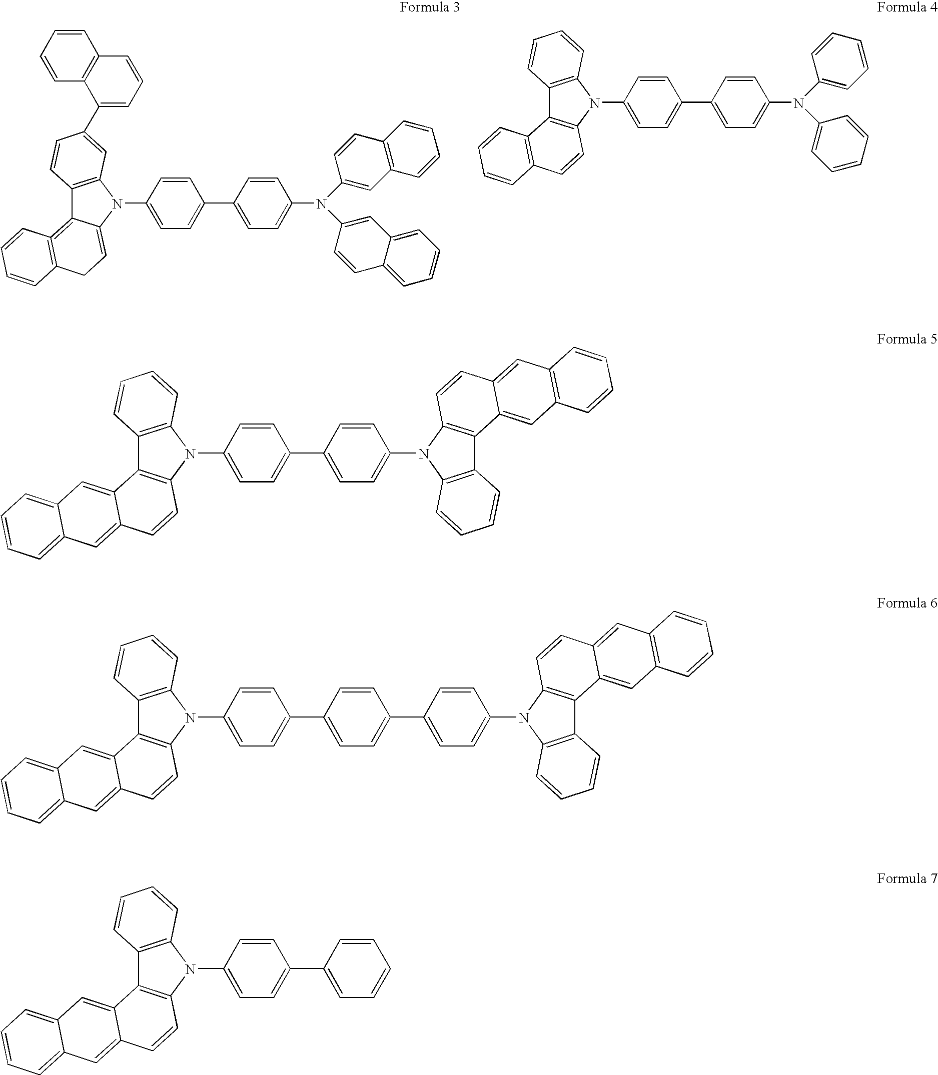

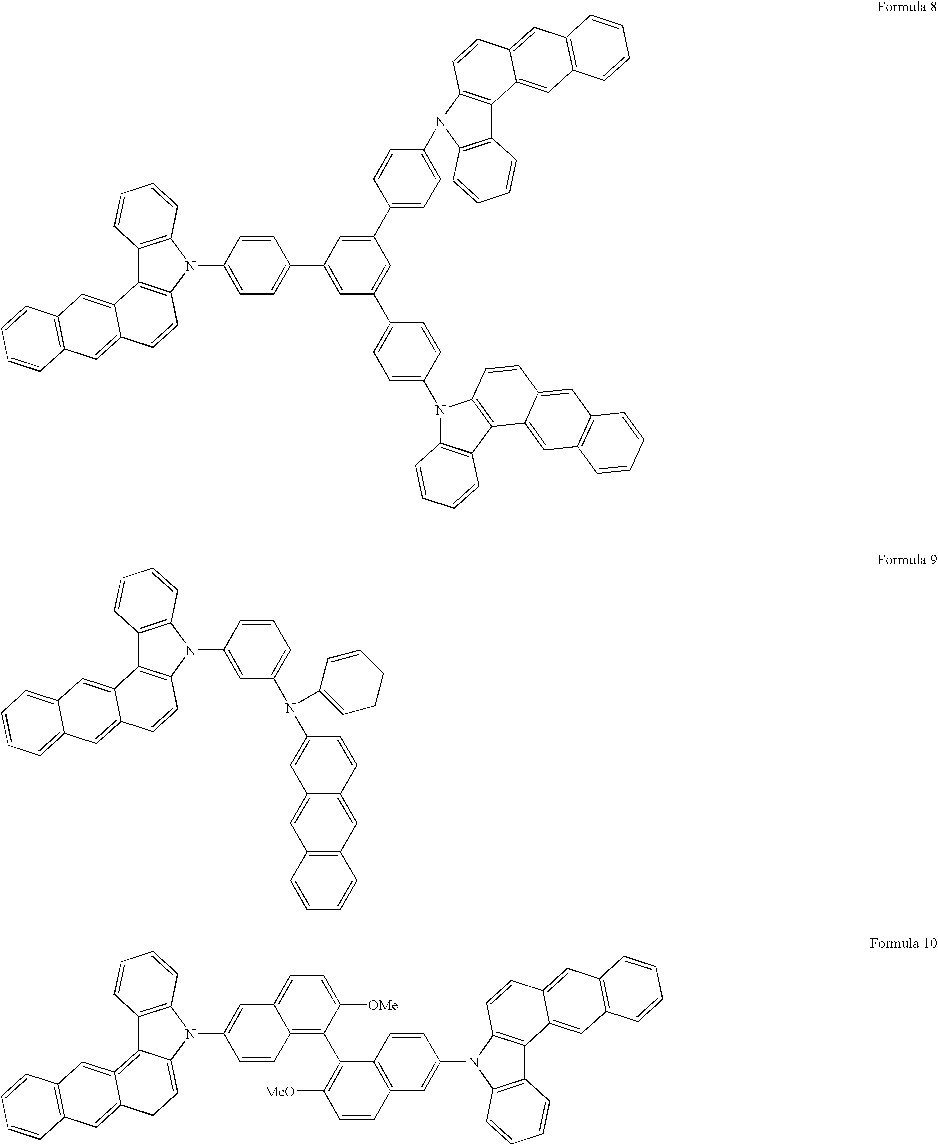

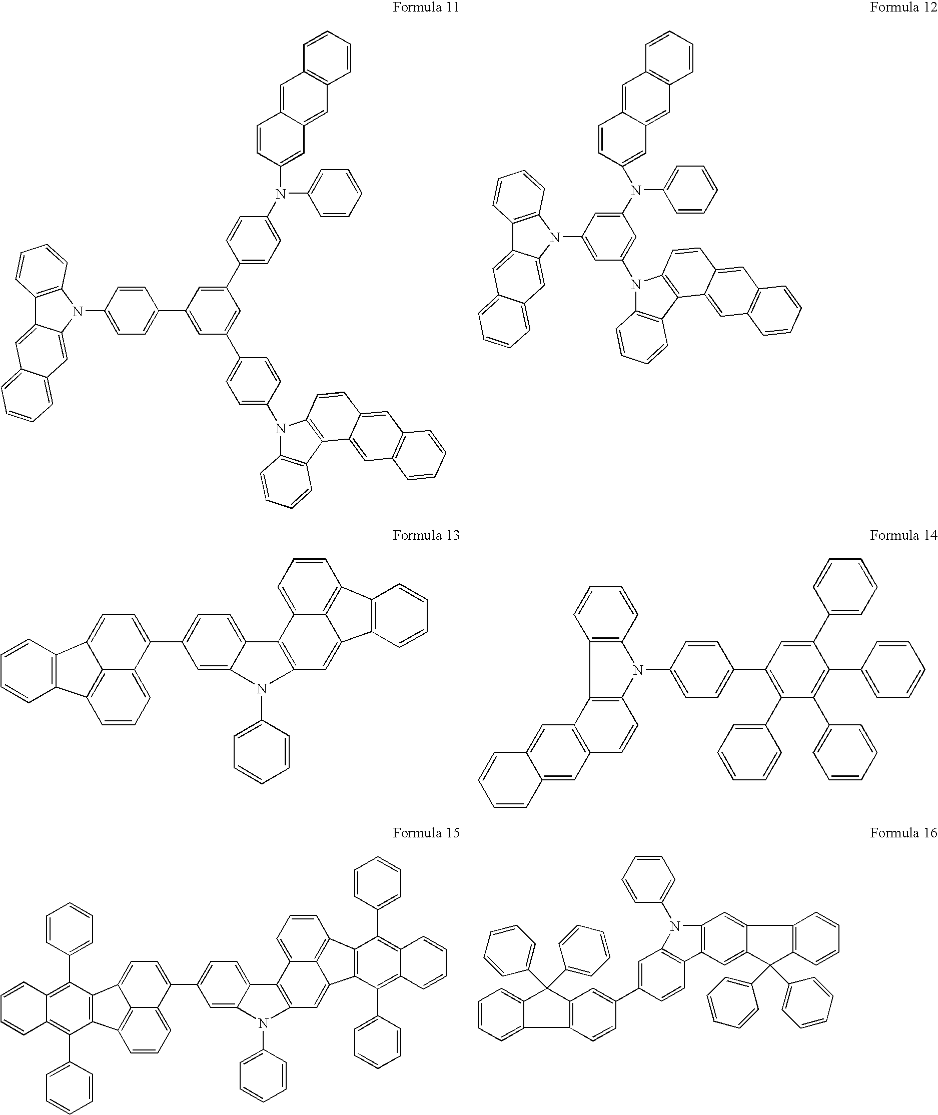

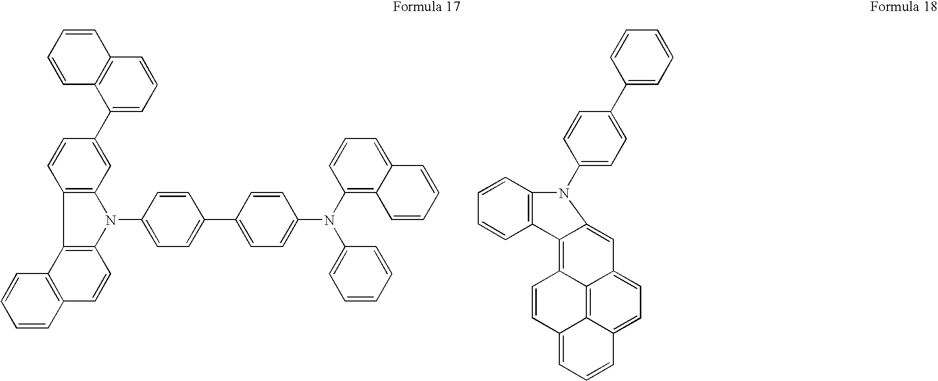

The organic light emitting compound represented by Formula 1 or 2 can be synthesized using conventional synthesis methods which will be described in more detail later with reference to reaction schemes of Synthesis Examples.

Thermal stability of the organic light emitting compounds represented by Formulae 1 through 18 can be determined by measuring the degradation temperature (T_{d}) and melting point (Tₘ) of the compounds through thermal analysis using thermo gravimetric analysis (“TGA”) and differential scanning calorimetry (“DSC”). For example, T_{d} and Tₘ of the organic light emitting compound represented by Formula 5 are 522° C. and 378° C., respectively. From the results, it can be seen that an organic light emitting compound having high thermal stability can be obtained using any of the organic light emitting compounds represented by Formulae 1 through 18. In addition, emitting ability of each of the organic light emitting compounds represented by Formulae 1 through 18 can be evaluated by measuring photoluminescence (PL) spectra of the compounds. For example, the organic light emitting compound represented by Formula 5 has a maximum wavelength of 460 nm in a solution, and CIE coordinate thereof is (0.14, 0.09). From the results, it can be seen that the organic light emitting compounds represented by Formulae 1 through 18 are blue light-emitting materials having high color purity.

According to an embodiment, there is provided an organic light emitting device comprising: a first electrode; a second electrode; and an organic layer interposed between the first electrode and the second electrode, wherein the organic layer includes at least one of organic light emitting compounds represented by Formula 1 and 2:

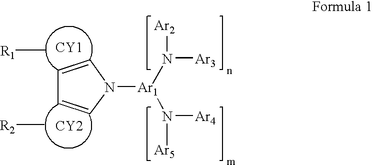

where CY1 and CY2 are each independently a fused C₆-C₅₀ aromatic ring;

Ar₁ is a substituted or unsubstituted C₆-C₅₀ arylene group;

Ar₂, Ar₃, Ar₄ and Ar₅ are each independently a substituted or unsubstituted C₆-C₅₀ aryl group, Ar₂ and Ar₃ are separate or bound to each other to form a substituted or unsubstituted C₁₃-C₁₀₀ heteroaryl group containing N, and Ar₄ and Ar₅ are separate or bound to each other to form a substituted or unsubstituted C₁₃-C₁₀₀ heteroaryl group containing N;

R₁ and R₂ represent one or more substituent groups and are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group;

n and m are each independently an integer of 0 through 3; and

wherein Ar₁ is a substituted or unsubstituted C₆-C₅₀ aryl group when n and m are both 0, and CY1 and CY2 are unsubstituted where R₁ and R₂ are each respectively a hydrogen;

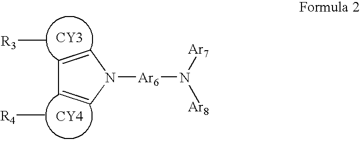

where CY3 and CY4 are each independently a fused benzene ring or a fused naphthalene ring;

Ar₆ is a substituted or unsubstituted C₆-C₅₀ arylene group;

Ar₇ and Ar₈ are each independently a substituted or unsubstituted C₆-C₅₀ aryl group, or a substituted or unsubstituted C₁₃-C₁₀₀ heteroaryl group;

R₃ and R₄ are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; and wherein, CY3 and CY4 are not simultaneously a fused benzene ring and CY3 and CY4 are unsubstituted where R₃ and R₄ are respectively each a hydrogen.

The organic light emitting compounds represented by Formula 1 and 2 are suitable for an organic layer of an organic light emitting device, in particular, an emission layer, a hole injection layer or a hole transport layer.

Unlike a conventional organic light emitting device that includes an organic layer having low stability, the organic light emitting device according to the embodiments herein can have low turn-on voltage, high efficiency, high color purity, high luminance, and the like, by including an organic light emitting compound that has good solubility and high thermal stability, and can form a stable organic layer, when manufactured using a solution coating method.

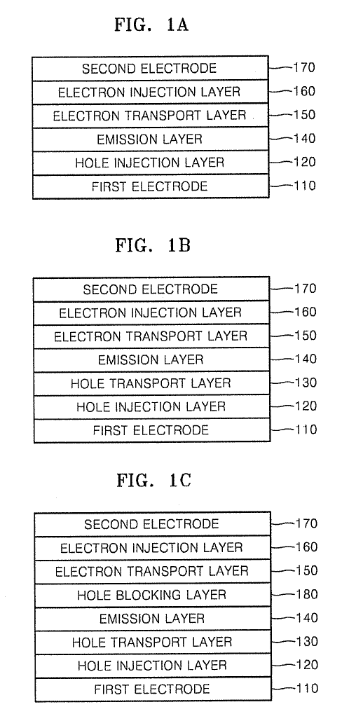

The organic light emitting device can have various structures. The organic light emitting device can further include at least one selected from the group consisting of a hole injection layer, a hole transport layer, a hole blocking layer, an electron blocking layer, an electron transport layer and an electron injection layer, between the first electrode and the second electrode. More specifically, FIGS. 1A through 1C are schematic cross-sectional views illustrating structures of the organic light emitting device according to an embodiment. Referring to FIG. 1A, the organic light emitting device has a first electrode 110/hole injection layer 120/emission layer 140/electron transport layer 150/electron injection layer 160/second electrode 170 structure. Referring to FIG. 1B, the organic light emitting device has a first electrode 110/hole injection layer 120/hole transport layer 130/emission layer 140/electron transport layer 150/electron injection layer 160/second electrode 170 structure. Referring to FIG. 1C, the organic light emitting device has a first electrode 110/hole injection layer 120/hole transport layer 130/emission layer 140/hole blocking layer 180/electron transport layer 150/electron injection layer 160/second electrode 170 structure. Here, at least one of the emission layer 140, the hole injection layer 120 and the hole transport layer 150 can include the organic light emitting compound disclosed herein.

The emission layer 140 of the organic light emitting device according to an embodiment may include a red, green, blue or white phosphorescent or fluorescent dopant. The phosphorescent dopant can be an organic metal compound which contains at least one of Ir, Pt, Os, Ti, Zr, Hf, Eu, Tb, and Tm.

In addition, the organic light emitting compound can be used as a fluorescent dopant on the emission layer 140.

Hereinafter, a method of manufacturing an organic light emitting device according to an embodiment will be described with reference to the organic light emitting device illustrated in FIG. 1C.

First, a first electrode 110 is formed by depositing or sputtering a high work-function material on a surface of a substrate (not shown). The first electrode 110 can be an anode. The substrate, which can be any substrate that is used in conventional organic light emitting devices, may be a glass substrate or a transparent plastic substrate that has excellent mechanical strength, thermal stability, transparency, and surface smoothness, can be easily treated, and is waterproof. The first electrode can be formed of indium tin oxide (“ITO”), indium zinc oxide (“IZO”), tin oxide (SnO₂), zinc oxide (ZnO), or any transparent material having high conductivity.

Then, a hole injection layer (“HIL”) 120 can be formed on a surface of the first electrode 110 opposite the substrate by vacuum deposition, spin coating, casting, Langmuir Blodgett (“LB”) deposition, or the like.

When the HIL 120 is formed by vacuum deposition, vacuum deposition conditions may vary according to the compound that is used to form the HIL 120, and the desired structure and thermal properties of the HIL 120 to be formed. In general, however, the vacuum deposition may be performed at a deposition temperature of 100° C. to 500° C., a pressure of 10⁻⁸ to 10³ torr, a deposition speed of 0.01 to 100 Å/sec, and to a layer thickness of 100 Å to 10 μm.

When the HIL 120 is formed by spin coating, coating conditions may vary according to the compound that is used to form the HIL 120, and the desired structure and thermal properties of the HIL 120 to be formed. In general, however, the coating speed may be in the range of about 2,000 to 5,000 rpm, and a temperature for heat treatment, which is performed to remove a solvent after coating, may be in the range of about 80 to 200° C.

A material used to form the HIL can be formed of the organic light emitting compound represented by Formula 1. For example, the material may be a phthalocyanine compound, such as copper phthalocyanine as disclosed in U.S. Pat. No. 4,356,429; a star-burst type amine derivative, such as 4,4′,4″-tris(N-carbazolyl)-triphenylamine (“TCTA”), 4,4′,4″-tris(3-methylphenylphenylamino)triphenylamine (“m-MTDATA”), or (“m-MTDATA”), and 1,3,4-tris{4-[methylphenyl(phenyl)amino]phenyl}benzene (“m-MTDAPB”), disclosed in Advanced Materials, 1994, vol. 6, p. 677; soluble and conductive polymer such as polyaniline/Dodecylbenzenesulfonic acid (“PANI/DBSA”); poly(3,4-ethylenedioxythiophene)/poly(4-styrenesulfonate) (“PEDOT/PSS”): polyaniline/camphor sulfonic acid (“Pani/CSA”); (polyaniline)/poly(4-styrenesulfonate) (“PANI/PSS”); or the like.

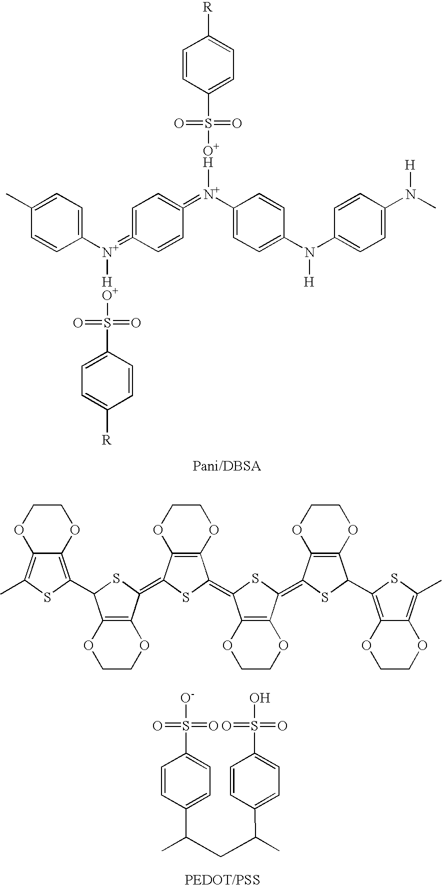

The thickness of the HIL 120 may be in the range of about 100 to about 10,000 Å, and specifically, in the range of about 100 to about 1,000 Å. When the thickness of the HIL 120 is less than about 100 Å, the hole injecting ability of the HIL 120 may be reduced. On the other hand, when the thickness of the HIL 120 is greater than about 10,000 Å, a turn-on voltage of the organic light emitting device can be increased. Then, a hole transport layer (“HTL”) 130 can be formed on the HIL 130 using vacuum deposition, spin coating, casting, LB, or the like. When the HTL 130 is formed by vacuum deposition or spin coating, the deposition and coating conditions are similar to those for the formation of the HIL 130, although the deposition and coating conditions may vary according to the material that is used to form the HTL 130.

A material used to form the HTL 130 can include the organic light emitting compound represented by Formula 1 described above. In addition, for example, the HTL 130 can be formed of a carbazole derivative, such as N-phenylcarbazole, polyvinylcarbazole; an amine derivative having an aromatic condensation ring such as N,N′-bis(3-methylphenyl)-N,N′-diphenyl-[1,1-biphenyl]-4,4′-diamine (“TPD”), N,N′-di(naphthalene-1-yl)-N,N′-diphenyl benzidine (“α-NPD”); or the like.

The thickness of the HTL 130 may be in the range of about 50 to about 1,000 Å, and specifically, about 100 to about 600 Å. Where the thickness of the HTL 130 is less than about 50 Å, the hole transporting ability of the HTL may be reduced. On the other hand, where the thickness of the HTL is greater than about 1,000 Å, the turn-on voltage of the organic light emitting device may increase.

Then, an emission layer (“EML”) 140 can be formed on a surface of the HTL 130 opposite HIL 120 by vacuum deposition, spin coating, casting, LB deposition, or the like. When the EML is formed by vacuum deposition or spin coating, the deposition and coating conditions are similar to those for the formation of the HIL 120, although the deposition and coating conditions may vary according to the material that is used to form the EML.

The emission layer 140 can include the organic light emitting compound represented by Formula 1 described above. Here, the emission layer 140 can be formed using a known host material or a known dopant material in company with the compound of Formula 1. The organic light emitting compound of Formula 1 can be used alone. The host material may be, for example, tris(8-quinolinolato)-aluminum (“Alq₃”), 4,4′-N,N′-dicarbazole-biphenyl (“CBP”), poly(n-vinylcarbazole) (“PVK”), or the like.

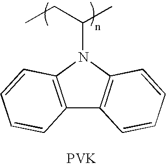

Exemplary dopant materials can include a fluorescent dopant can include IDE102 and IDE105 obtained from Idemitsu Co., C545T obtained from Hiyasibara Co., and the like. Exemplary phosphorescent dopants include a red phosphorescent dopant such as platinum octatethyl porphine (“PtOEP”), RD 61 obtained from UDC Co., a green phosphorescent dopant such as Ir(PPy)₃ (PPy=2-phenylpyridine), a blue phosphorescent dopant such as iridium (III) bis[4,6-di-fluorophenyl)-pyridinato-N,C²′]picolinate (referred to herein as (“F₂Irpic”)), and the like. The concentration of the dopant is not limited, but is conventionally in the range of 0.01 to 15 parts by weight based on 100 parts by weight of a host.

The thickness of the EML 140 may be in the range of about 100 to about 1,000 Å, and specifically, about 200 to about 600 Å. When the thickness of the EML is less than about 100 Å, the emissive ability of the EML 140 may be reduced. On the other hand, when the thickness of the EML 140 is greater than about 1,000 Å, the turn-on voltage of the organic light emitting device may increase. When the EML 140 includes a phosphorous dopant, a hole blocking layer (“HBL”) 180 can be formed on a surface of the EML 140 opposite HTL 130 by vacuum deposition, spin coating, casting, LB deposition, or the like in order to prevent triplet excitons or holes from migrating into an electron transport layer (“ETL”) 150. When the HBL 180 is formed by vacuum deposition or spin coating, the deposition and coating conditions are similar to those for the formation of the HIL 120, although deposition and coating conditions may vary according to the material that is used to form the HBL 180. Examples of the material used to form the HBL 180 include oxadiazole derivatives, triazole derivatives, phenanthroline derivatives such as for example 2,9-dimethyl-4,7-diphenyl-1,10-phenanthroline (“BCP”), and the like.

The thickness of the HBL may be in the range of about 50 to about 1,000 Å, and specifically, about 100 to about 300 Å. When the thickness of the HBL is less than about 50 Å, the hole blocking ability of the HBL 180 may be reduced. On the other hand, when the thickness of the HBL 180 is greater than about 1,000 Å, the turn-on voltage of the organic light emitting device may increase.

Then, an electron transport layer (ETL) 150 is formed on a surface of HBL 180 opposite EML 140 by vacuum deposition, spin coating, casting, or the like. When the ETL 150 is formed by vacuum deposition or spin coating, the deposition and coating conditions are, in general, similar to those for the formation of the HIL 120, although the deposition and coating conditions may vary according to the material that is used to form the ETL 150. The ETL 150 transports electrons injected from the cathode, and the ETL 150 may be formed of a quinoline derivative, in particular, tris(8-quinolinorate)aluminum (“Alq₃”), TAZ (see below), bis(2-methyl-8-quinolinolato)-aluminum biphenolate (“Balq”), or the like, which is known in the art.

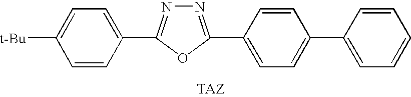

The thickness of the ETL 150 may be in the range of about 100 to about 1,000 Å, and specifically, about 200 to about 500 Å. When the thickness of the ETL 150 is less than about 100 Å, the electron transporting ability of the ETL 150 may be reduced. On the other hand, when the thickness of the ETL 150 is greater than about 1,000 Å, the turn-on voltage of the organic light emitting device may increase.

In addition, an electron injection layer (“EIL”) 160 that makes it easy for electrons to be injected from a cathode may be formed on a surface of the ETL 150 opposite HBL 180. A material used to form the EIL 160 is not particularly limited. The EIL 160 may be formed of LiF, NaCl, CsF, Li₂O, BaO, or the like, materials which are known in the art. Conditions for the deposition of the EIL 160 are, in general, similar to conditions for the formation of the HIL 120, although they may vary according to the material that is used to form the EIL 160.

The thickness of the EIL 160 may be in the range of about 1 to about 100 Å, and specifically, about 5 to about 50 Å. When the thickness of the EIL 160 is less than about 1 Å, the electron injecting ability of the EIL 160 may be reduced. On the other hand, when the thickness of the EIL 160 is greater than about 100 Å, the turn-on voltage of the organic light emitting device may increase. Finally, a second electrode 170 can be formed on a surface of the EIL 160 opposite ETL 150 by vacuum deposition, sputtering, or the like. The second electrode 170 can be used as a cathode. The second electrode may be formed of a low work-function metal, an alloy, an electrically conductive compound, or a combination thereof. In particular, the second electrode may be formed of Li, Mg, Al, Al—Li, Ca, Mg—In, Mg—Ag, or the like. Alternatively, a transparent cathode formed of ITO or IZO can be used to produce a front surface light emitting device.

There is provided a method of manufacturing an organic light emitting device according to an embodiment including: forming a first electrode; forming an organic thin film on a surface of the first electrode including the organic light emitting compound represented by any one of Formulae 1 through 18 on the first electrode; and forming a second electrode on a surface of the organic thin film opposite the first electrode. A step of sintering the organic thin film can be performed before forming the second electrode. The organic thin film may be formed using a wet spinning method including solution deposition, spin coating, inkjet printing and spray printing or a heat transfer method.

Hereinafter, synthesis examples and examples of organic light emitting compounds according to embodiments will be described in detail. However, the synthesis examples and examples are provided to facilitate the understanding of the present invention only, and are not intended to limit the scope of the present invention.

### EXAMPLES

### Synthesis Example 1

Compound 3, represented by Formula 3, was synthesized according to Reaction Schemes 1, 2 and 3:

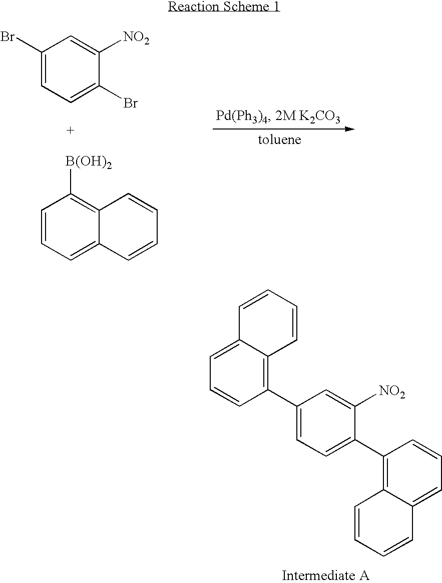

Synthesis of Intermediate A

8.4 g of 2,5-dibromonitrobenzene (30 mmol), 10.8 g of 1-naphthaleneboronic acid (62.6 mmol), 520 mg of tetrakis triphenylphosphine palladium (Pd(PPh₃)₄) (0.45 mmol) and 63 ml of a 2M aqueous potassium carbonate solution (126 mmol) were dissolved in 100 ml of toluene, respectively, and then the mixtures were added to a 500 ml round bottom flask under argon gas. Then, the mixture was refluxed for 24 hours. After the reaction was terminated, a solvent was removed by evaporation. Then, the residue washed with 500 ml of ethyl acetate and 500 ml of water. Thereafter, an organic layer was collected and dried over anhydrous magnesium sulfate. Subsequently, the dried organic layer was purified using silica chromatography to obtain 9.5 g of a compound represented by Intermediate A (yield 84%).

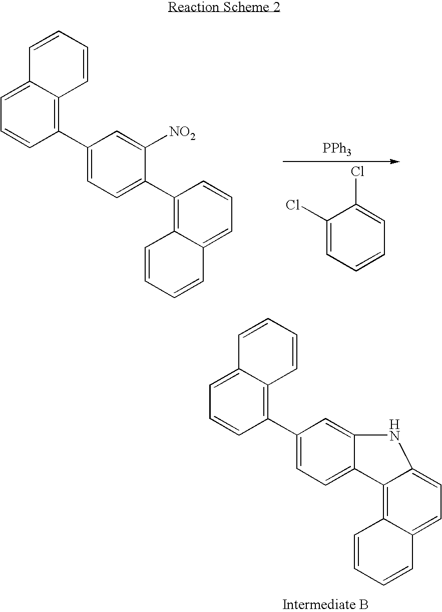

Synthesis of Intermediate B

8.0 g of Intermediate A (21.3 mmol) and 14 g of triphenylphosphine (PPh₃) (53.3 mmol) were dissolved in 42 ml of 1,2-dichlorobenzene, and the mixture was added to a 500 ml round bottom flask and then refluxed for 24 hours. After the reaction was terminated, the reactant was purified using silica chromatography to obtain 4.1 g of a compound represented by Intermediate B (yield 56%).

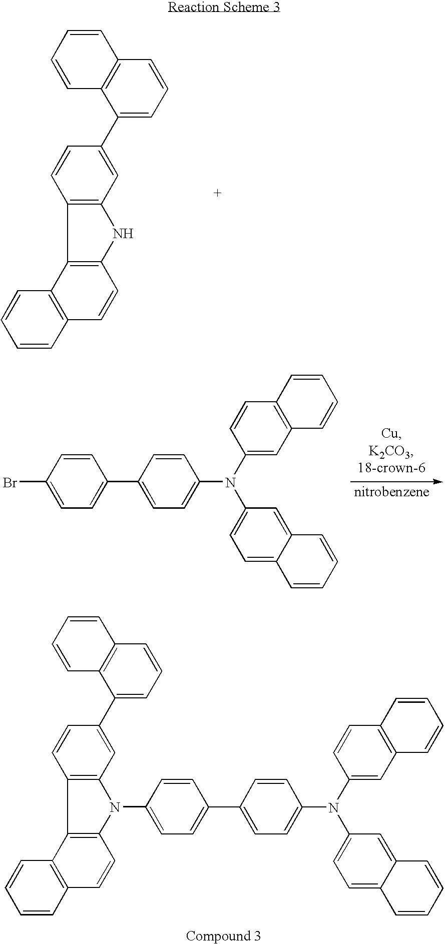

Synthesis of Compound 3

370 mg of Intermediate B (1 mmol), 137 mg of copper (2.2 mmol), 597 mg of potassium carbonate (4.3 mmol), 86 mg of 18-crown-6-ether (0.32 mmol), 650 mg of N-(4-bromobiphenyl-4-yl)-N-(naphthalene-2-yl)naphthalene-2-amine (1.3 mmol) were dissolved in 3 ml of nitrobenzene, and the mixture was added to a 500 ml round bottom flask and then refluxed for 24 hours. After the reaction was terminated, the solvent was evaporated and thereby removed. Then, the residue washed with 50 ml of ethyl acetate and 50 ml of water. Thereafter, an organic layer was collected and dried with magnesium sulfate. Subsequently, the dried organic layer was purified using silica chromatography to obtain 403 mg of a compound represented by Compound 3 (yield 53%).

¹H-NMR (CDCl₃, 300 MHz, ppm): 8.7-6.9 (m, 38H).

### Synthesis Example 2

Compound 5 represented by Formula 5 was synthesized according to Reaction Schemes 4, 5 and 6:

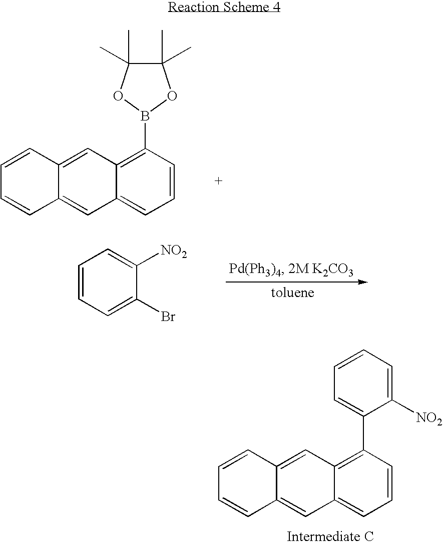

Synthesis of Intermediate C

729 mg of 2-bromonitrobenzene (3.6 mmol), 1.1 g of 1-(4,4,5,5-tetramethyl-1,3,2-dioxaborolan-2-yl)anthracene (3.6 mmol), 63 mg of tetrakis triphenylphosphine palladium (Pd(PPh₃)₄) (0.054 mmol) and 3.8 ml of a 2M aqueous potassium carbonate (K₂CO₃) (7.6 mmol) were dissolved in 12 ml of toluene, respectively, and the mixtures were added to a 500 ml round bottom flask and then refluxed for 24 hours. After the reaction was terminated, the solvent was evaporated and thereby removed. Then, the residue washed with 100 ml of ethyl acetate and 100 ml of water. Thereafter, an organic layer was collected and dried with magnesium sulfate. Subsequently, the dried organic layer was purified using silica chromatography to obtain 880 mg of a compound represented by Intermediate C (yield 81%).

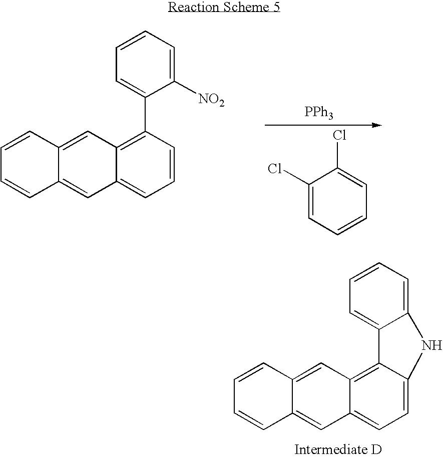

Synthesis of Intermediate D

806 mg of Intermediate C (2.7 mmol) and 1.8 g of triphenylphosphine (PPh₃) (6.7 mmol) were dissolved in 5.4 ml of 1,2-dichlorobenzene, and the mixtures were added to a 500 ml round bottom flask and then refluxed for 24 hours. After the reaction was terminated, the reactant was purified using silica chromatography to obtain 0.4 mg of a compound represented by Intermediate D (yield 56%).

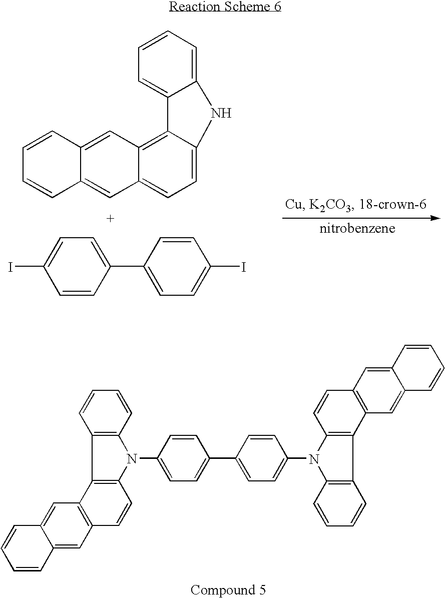

Synthesis of Compound 5

420 mg of Intermediate D (1.5 mmol), 132 mg of copper (2.0 mmol), 575 mg of potassium carbonate (4.2 mmol), 42 mg of 18-crown-6-ether (0.16 mmol), 211 mg of 4,4-diiodobiphenyl (0.5 mmol) were dissolved in 3 ml of nitrobenzene, and the mixtures were added to a 500 ml round bottom flask and then refluxed for 24 hours. After the reaction was terminated, the solvent was evaporated and thereby removed. Then, the residue washed with 50 ml of ethyl acetate and 50 ml of water. Thereafter, an organic layer was collected and was dried with magnesium sulfate. Subsequently, the dried organic layer was purified using silica chromatography to obtain 220 mg of a compound represented by Compound 5 (yield 62%).

¹H-NMR (CDCl₃, 300 MHz, ppm): 9.3-6.9 (m, 32H).

### Synthesis Example 3

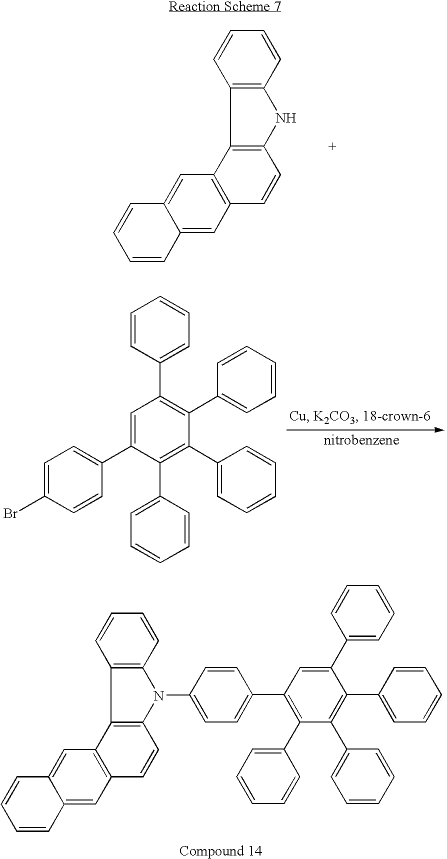

Synthesis of Compound 14

840 mg of Intermediate D (3 mmol), 260 mg of copper (4.0 mmol), 1.2 g of potassium carbonate (8.4 mmol), 85 mg of 18-crown-6-ether (0.32 mmol), 540 mg of 2,3,4,5-tetraphenyl-4-bromophenyl (1 mmol) were dissolved in 6 ml of nitrobenzene, and the mixtures were added to a 500 ml round bottom flask and then refluxed for 24 hours. After the reaction was terminated, the solvent was evaporated and thereby removed. Then, the residue washed with 100 ml of ethyl acetate and 100 ml of water. Thereafter, an organic layer was collected and dried with magnesium sulfate. Subsequently, the dried organic layer was purified by silica chromatography to obtain 490 mg of a compound represented by Compound 14 (yield 67%).

¹H-NMR (CDCl₃, 300 MHz, ppm): 9.3-6.9 (m, 37H).

### Evaluation Example 1

### Evaluation of Emitting Ability of Compound (Solution State)

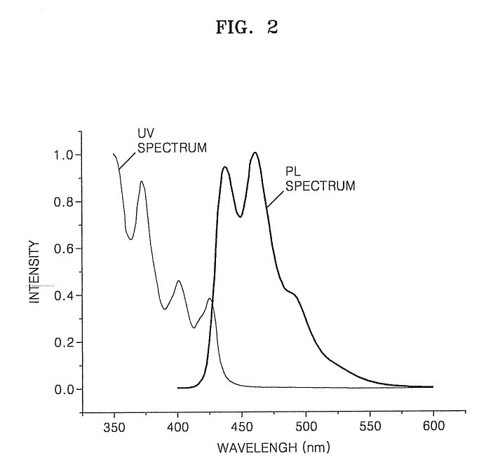

Emitting ability of each compound was evaluated by measuring photoluminescence (PL) spectra of the compounds. First, Compound 3 was diluted to a concentration of 10 mM in toluene. Then, a photoluminescence (PL) spectrum of the compound was measured using an ISC PC1 spectrofluorometer in which a Xenon lamp was installed. These processes were repeated with respect to Compounds 5 and 14. The results are shown in Table 1 below. In particular, FIG. 2 is a graph illustrating UV and photoluminescence (PL) spectra of a solution comprising Compound 5.

TABLE 1

| Compound No. | Maximum PL wavelength (nm) |
|---|---|
| 3 | 410 |
| 5 | 460 |
| 14 | 440 |

From the results, it can be seen that organic light emitting compounds according to embodiments of the present invention have light emitting properties suitable for an organic light emitting device.

### Example 1

Using Compound 3 as a dopant of an emission layer, an organic light emitting device having the following structure was manufactured: ITO/α-NPD (500 Å)/Compound 3+ADN (500 Å)/Alq3 (200 Å)/LiF (10 Å)/Al (2,000 Å). As an anode, a 15 Ω/cm² (1000 Å) ITO glass substrate was cut to a size of 50 mm×50 mm×0.7 mm, microwave washed with isopropyl alcohol and pure water for 15 minutes each, respectively, and then washed with UV ozone for 30 minutes. N,N′-di(naphthalene-1-yl)-N,N′-diphenyl benzidene (α-NPD) was vacuum deposited on the substrate to form a hole injection layer having a thickness of 500 Å. Compound 3 and 9,10-di(naphthalene-2-yl)anthracene (ADN) (3 volume parts of Compound 3 per 100 volume parts of ADN 100) represented by Formula 19 below were vacuum deposited to form an emission layer with a thickness of 500 Å. Then, Alq3 was vacuum deposited on the emission layer to form an electron transport layer having a thickness of 200 Å. 10 Å of LiF and 2,000 Å of Al were sequentially vacuum deposited on the electron transport layer to form an electron injection layer and a cathode, respectively. Accordingly, an organic light emitting device having the structure illustrated in FIG. 1A was manufactured. This organic light emitting device is referred to as Sample 1.

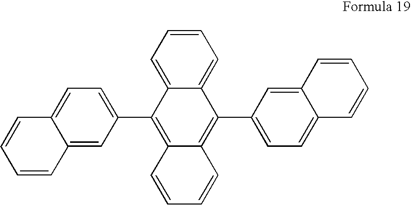

### Example 2

An organic light emitting device having a structure of ITO/α-NPD (500 Å)/Compound 5+ADN (500 Å)/Alq3 (200 Å)/LiF (10 Å)/Al (2,000 Å) was manufactured in the same manner as in Example 1, except that Compound 5 was used as a dopant instead of Compound 3. This organic light emitting device is referred to as Sample 2.

### Example 3

An organic light emitting device having a layered structure of ITO/α-NPD (500 Å)/Compound 14+ADN (500 Å)/Alq3 (200 Å)/LiF (10 Å)/Al (2,000 Å) was manufactured in the same manner as in Example 1, except that Compound 14 was used as a dopant instead of Compound 3. This organic light emitting device is referred to as Sample 3.

### Evaluation Example 2

### Evaluation of Properties of Samples 1, 2, and 3

Turn-on voltage, luminance and efficiency of Samples 1, 2 and 3 were measured using a PR650 (Spectroscan) Source Measurement Unit, respectively. The results are shown in Table 4 below, where efficiency is reported in units of lumens per watt (lm/W) and luminance is reported in units of candles per square meter (cd/m²).

TABLE 2

| Sample | | | |
|---|---|---|---|
| No. | Turn-on voltage (V) | Efficiency (lm/W) | luminance (cd/m²) |
| 1 | 3.8 | 2.9 | 6,542 |
| 2 | 3.4 | 3.1 | 7,102 |
| 3 | 3.4 | 3.0 | 6,983 |

From the results shown in Table 2, it can be seen that Samples 1 through 3 have good turn-on voltage as a desirable electrical property.

The organic light emitting compounds represented by Formulae 1 through 3 have good solubility, good light emitting properties, and high thermal stability. Therefore, an organic light emitting device manufactured using any of the organic light emitting compounds disclosed herein has low turn-on voltage, high luminance and high efficiency.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An organic light emitting compound represented by Formula 1 below: 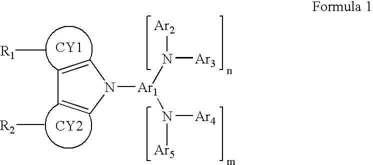
where CY1 and CY2 are each independently a fused C₆-C₅₀ aromatic ring;
Ar₁ is a substituted or unsubstituted C₆-C₅₀ arylene group;
Ar₂, Ar₃, Ar₄ and Ar₅ are each independently a substituted or unsubstituted C₆-C₅₀ aryl group, Ar₂ and Ar₃ are separate or bound to each other to form a substituted or unsubstituted C₁₃-C₁₀₀ heteroaryl group containing N, and Ar₄ and Ar₅ are separate or bound to each other to form a substituted or unsubstituted C₁₃-C₁₀₀ heteroaryl group containing N;
R₁ and R₂ represent one or more substituent groups and are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; n and m are each independently an integer of 0 through 3; and
wherein Ar₁ is a substituted or unsubstituted C₆-C₅₀ aryl group when n and m are both 0, and CY1 and CY2 are unsubstituted where R₁ and R₂ are each respectively a hydrogen.

2. The organic light emitting compound of claim 1, represented by Formula 2 below: 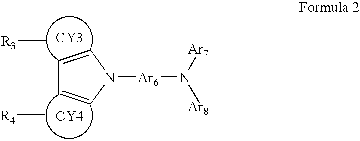
where CY3 and CY4 are each independently a fused benzene ring or a fused naphthalene ring;
Ar₆ is a substituted or unsubstituted C₆-C₅₀ arylene group;
Ar₇ and Ar₈ are each independently a substituted or unsubstituted C₆-C₅₀ aryl group or a substituted or unsubstituted C₁₃-C₁₀₀ heteroaryl group; R₁ and R₂ represent one or more substituent groups and are each independently a hydrogen atom, a halogen atom, a cyano group, a nitro group, a hydroxyl group, a substituted or unsubstituted C₁-C₅₀ alkyl group, a substituted or unsubstituted C₁-C₅₀ alkoxy group, a substituted or unsubstituted C₅-C₅₀ cycloalkyl group, a substituted or unsubstituted C₅-C₅₀ heterocycloalkyl group, a substituted or unsubstituted C₆-C₅₀ aryl group, a substituted or unsubstituted C₂-C₅₀ heteroaryl group, or —N(Z₁)(Z₂) or —Si(Z₃)(Z₄)(Z₅) where Z₁, Z₂, Z₃, Z₄ and Z₅ are each independently a hydrogen atom, a substituted or unsubstituted C₁-C₅₀ alkyl group, a substituted or unsubstituted C₆-C₅₀ aryl group, a substituted or unsubstituted C₂-C₅₀ heteroaryl group, a substituted or unsubstituted C₅-C₅₀ cycloalkyl group or a substituted or unsubstituted C₅-C₅₀ heterocycloalkyl group; and
wherein, CY3 and CY4 are not simultaneously fused benzene rings, and CY3 and CY4 are unsubstituted where R₃ and R₄ are respectively a hydrogen.

3. The organic light emitting compound of claim 1, wherein substituents of the alkyl group, the alkoxy group, the arylene group, the aryl group, the heteroaryl group, the cycloalkyl group and the heterocycloalkyl group are at least one selected from the group consisting of —F; —Cl; —Br; —CN; —NO₂; —OH; an unsubstituted C₁-C₅₀ alkyl group or a C₁-C₅₀ alkyl group substituted with —F, —Cl, —Br, —CN, —NO₂ or —OH; an unsubstituted C₁-C₅₀ alkoxy group or a C₁-C₅₀ alkoxy group substituted with —F, —Cl, —Br, —CN, —NO₂ or —OH; an unsubstituted C₆-C₅₀ aryl group or a C₆-C₅₀ aryl group substituted with a C₁-C₅₀ alkyl group, a C₁-C₅₀ alkoxy group, —F, —Cl, —Br, —CN, —NO₂ or —OH; an unsubstituted C₂-C₅₀ heteroaryl group or a C₂-C₅₀ heteroaryl group substituted with a C₁-C₅₀ alkyl group, a C₁-C₅₀ alkoxy group, —F, —Cl, —Br, —CN, —NO₂ or —OH; an unsubstituted C₅-C₅₀ cycloalkyl group or a C₅-C₅₀ cycloalkyl group substituted with a C₁-C₅₀ alkyl group, a C₁-C₅₀ alkoxy group, —F, —Cl, —Br, —CN, —NO₂ or —OH; an unsubstituted C₅-C₅₀ heterocycloalkyl group or a C₅-C₅₀ heterocycloalkyl group substituted with a C₁-C₅₀ alkyl group, a C₁-C₅₀ alkoxy group, —F, —Cl, —Br, —CN, —NO₂ or —OH; and —N(Z₉)(Z₁₀) where Z₉ and Z₁₀ are each independently a hydrogen atom, a C₁-C₅₀ alkyl group, or a C₆-C₅₀ aryl group substituted with a C₁-C₅₀ alkyl group.

4. The organic light emitting compound of claim 2, wherein substituents of the alkyl group, the alkoxy group, the arylene group, the aryl group, the heteroaryl group, the cycloalkyl group and the heterocycloalkyl group are at least one selected from the group consisting of —F; —Cl; —Br; —CN; —NO₂; —OH; an unsubstituted C₁-C₅₀ alkyl group or a C₁-C₅₀ alkyl group substituted with —F, —Cl, —Br, —CN, —NO₂ or —OH; an unsubstituted C₁-C₅₀ alkoxy group or a C₁-C₅₀ alkoxy group substituted with —F, —Cl, —Br, —CN, —NO₂ or —OH; an unsubstituted C₅-C₅₀ aryl group or a C₅-C₅₀ aryl group substituted with a C₁-C₅₀ alkyl group, a C₁-C₅₀ alkoxy group, —F, —Cl, —Br, —CN, —NO₂ or —OH; an unsubstituted C₂-C₅₀ heteroaryl group or a C₂-C₅₀ heteroaryl group substituted with a C₁-C₅₀ alkyl group, a C₁-C₅₀ alkoxy group, —F, —Cl, —Br, —CN, —NO₂ or —OH; an unsubstituted C₅-C₅₀ cycloalkyl group or a C₅-C₅₀ cycloalkyl group substituted with a C₁-C₅₀ alkyl group, a C₁-C₅₀ alkoxy group, —F, —Cl, —Br, —CN, —NO₂ or —OH; an unsubstituted C₅-C₅₀ heterocycloalkyl group or a C₅-C₅₀ heterocycloalkyl group substituted with a C₁-C₅₀ alkyl group, a C₁-C₅₀ alkoxy group, —F, —Cl, —Br, —CN, —NO₂ or —OH; and —N(Z₉)(Z₁₀) where Z₉ and Z₁₀ are each independently a hydrogen atom, a C₁-C₅₀ alkyl group, or a C₆-C₅₀ aryl group substituted with a C₁-C₅₀ alkyl group.

5. The organic light emitting compound of claim 1, wherein CY1 is selected from the group consisting of a benzene ring, a toluene ring, a pentalene ring, an indene ring, a naphthalene ring, a biphenylene ring, an anthracene ring, an azulene ring, a heptalene ring, an acenaphthylene ring, a phenalene ring, a fluorene ring, a tetracene ring, a triphenylene ring, a pyrene ring, a chrysene ring, an ethyl-chrysene ring, a phycene ring, a perylene ring, a pentaphene ring, a pentacene ring, a tetraphenylene ring, a hexaphene ring, a hexacene ring, a rubicene ring, a coronene ring, a trinaphthylene ring, a heptaphene ring, a heptacene ring, a pyranthrene ring, an ovalene ring, a fluoranthrene ring, a benzofluoranthrene ring and derivatives thereof.

6. The organic light emitting compound of claim 1, wherein CY2 is selected from the group consisting of a pentalene ring, an indene ring, an anthracene ring, an azulene ring, a heptalene ring, an acenaphthylene ring, a phenalene ring, a fluorene ring, a tetracene ring, a triphenylene ring, a pyrene ring, a chrysene ring, an ethyl-chrysene ring, a phycene ring, a perylene ring, a pentaphene ring, a pentacene ring, a tetraphenylene ring, a hexaphene ring, a hexacene ring, a rubicene ring, a coronene ring, a trinaphthylene ring, a heptaphene ring, a heptacene ring, a pyranthrene ring, an ovalene ring, a fluoranthrene ring, a benzofluoranthrene ring and derivatives thereof.

7. The organic light emitting compound of claim 1, wherein Ar₂, Ar₃, Ar₄, and Ar₅ and Ar₁ where n=0 and m=0, are each independently selected from the group consisting of a phenyl group, a tolyl group, a biphenyl group, a pentarenyl group, an indenyl group, a naphthyl group, a biphenylenyl group, an anthracenyl group, an azulenyl group, a heptalenyl group, an acenaphthylenyl group, a phenalenyl group, a fluorenyl group, a methylanthryl group, a phenanthrenyl group, a triphenylene group, a pyrenyl group, a chrysenyl group, an ethyl-chrysenyl group, a pycenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovarenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolynyl group, a oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a di(C₆-C₅₀ aryl)aminophenyl group and derivatives thereof.

8. The organic light emitting compound of claim 1, wherein Ar₁ is selected from the group consisting of a phenylene group, a biphenylene group, a p-terphenylene group, a 1,3,5-triphenylbenzylene group, a tolylene group, a biphenylene group, a pentalenylene group, an indenylene group, a naphthylene group, a biphenylenylene group, an anthracenylene group, an azulenylene group, a heptalenylene group, an acenaphthylenylene group, a phenalenylene group, a fluorenylene group, a methylanthrylene group, a phenanthrenylene group, a triphenylenylene group, a pyrenylene group, a chrysenylene group, an ethyl-chrysenylene group, a picenylene group, a perylenylene group, a chloroperylenylene group, a pentaphenylene group, a pentacenylene group, a tetraphenylenylene group, a hexaphenylene group, a hexacenylene group, a rubicenylene group, a coronenylene group, a trinaphthylenylene group, a heptaphenylene group, a heptacenylene group, a pyranthrenylene group, an ovalenylene group, a carbazolylene group, a thiophenylene group, an indolylene group, a purinylene group, a benzimidazolylene group, a quinolinylene group, a benzothiophenylene group, a parathiazinylene group, a pyrrolylene group, a pyrazolylene group, an imidazolylene group, an imidazolinylene group, a oxazolylene group, a thiazolylene group, a triazolylene group, a tetrazolylene group, an oxadiazolylene group, a pyridinylene group, a pyridazinylene group, a pyrimidinylene group, a pyrazinylene group, a thianthrenylene group, a di(C₆-C₅₀ aryl)aminophenylene group and derivatives thereof.

9. The organic light emitting compound of claim 2, wherein Ar₇, and Ar₈ are each independently selected from the group consisting of a phenyl group, a tolyl group, a biphenyl group, a pentarenyl group, an indenyl group, a naphthyl group, a biphenylenyl group, an anthracenyl group, an azulenyl group, a heptalenyl group, an acenaphthylenyl group, a phenalenyl group, a fluorenyl group, a methylanthryl group, a phenanthrenyl group, a triphenylene group, a pyrenyl group, a chrysenyl group, an ethyl-chrysenyl group, a pycenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovarenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolynyl group, a oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a di(C₆-C₅₀ aryl)aminophenyl group and derivatives thereof.

10. The organic light emitting compound of claim 2, wherein Ar₆ is selected from the group consisting of a phenylene group, a biphenylene group, a p-terphenylene group, a 1,3,5-triphenylbenzylene group, a tolylene group, a biphenylene group, a pentalenylene group, an indenylene group, a naphthylene group, a biphenylenylene group, an anthracenylene group, an azulenylene group, a heptalenylene group, an acenaphthylenylene group, a phenalenylene group, a fluolenylene group, a methylanthrylene group, a phenanthrenylene group, a triphenylenylene group, a pyrenylene group, a chrysenylene group, an ethyl-chrysenylene group, a picenylene group, a perylenylene group, a chloroperylenylene group, a pentaphenylene group, a pentacenylene group, a tetraphenylenylene group, a hexaphenylene group, a hexacenylene group, a rubicenylene group, a coronenylene group, a trinaphthylenylene group, a heptaphenylene group, a heptacenylene group, a fluorenylene group, a pyranthrenylene group, an ovalenylene group, a carbazolylene group, a thiophenylene group, an indolylene group, a purinylene group, a benzimidazolylene group, a quinolinylene group, a benzothiophenylene group, a parathiazinylene group, a pyrrolylene group, a pyrazolylene group, an imidazolylene group, an imidazolinylene group, a oxazolylene group, a thiazolylene group, a triazolylene group, a tetrazolylene group, an oxadiazolylene group, a pyridinylene group, a pyridazinylene group, a pyrimidinylene group, a pyrazinylene group, a thianthrenylene group, a di(C₆-C₅₀ aryl)aminophenylene group and derivatives thereof.

11. The organic light emitting compound of claim 1, wherein R₁ and R₂, R₃ and R₄ are each independently selected from the group consisting of a hydrogen atom, a C₁-C₅₀ alkyl group, a C₁-C₅₀ alkoxy group, a phenyl group, a tolyl group, a biphenyl group, a pentarenyl group, an indenyl group, a naphthyl group, a biphenylenyl group, an anthracenyl group, an azulenyl group, a heptalenyl group, an acenaphthylenyl group, a phenarenyl group, a fluolenyl group, a methylanthryl group, a phenanthrenyl group, a triphenylenyl group, a pyrenyl group, a chrysenyl group, an ethyl-chrysenyl group, a pycenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovarenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolynyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolidinyl group, a piperidinyl group, a piperazinyl group, a morpholinyl group, a di(C₆-C₅₀ aryl)amino group, a di(C₆-C₅₀ aryl)aminophenyl group, a tri(C₆-C₅₀ aryl)silyl group and derivatives thereof.

12. The organic light emitting compound of claim 2, wherein R₃ and R₄ are each independently selected from the group consisting of a hydrogen atom, a C₁-C₅₀ alkyl group, a C₁-C₅₀ alkoxy group, a phenyl group, a tolyl group, a biphenyl group, a pentarenyl group, an indenyl group, a naphthyl group, a biphenylenyl group, an anthracenyl group, an azulenyl group, a heptalenyl group, an acenaphthylenyl group, a phenarenyl group, a fluolenyl group, a methylanthryl group, a phenanthrenyl group, a triphenylenyl group, a pyrenyl group, a chrysenyl group, an ethyl-chrysenyl group, a pycenyl group, a perylenyl group, a chloroperylenyl group, a pentaphenyl group, a pentacenyl group, a tetraphenylenyl group, a hexaphenyl group, a hexacenyl group, a rubicenyl group, a coronenyl group, a trinaphthylenyl group, a heptaphenyl group, a heptacenyl group, a fluorenyl group, a pyranthrenyl group, an ovarenyl group, a carbazolyl group, a thiophenyl group, an indolyl group, a purinyl group, a benzimidazolyl group, a quinolinyl group, a benzothiophenyl group, a parathiazinyl group, a pyrrolyl group, a pyrazolyl group, an imidazolyl group, an imidazolynyl group, an oxazolyl group, a thiazolyl group, a triazolyl group, a tetrazolyl group, an oxadiazolyl group, a pyridinyl group, a pyridazinyl group, a pyrimidinyl group, a pyrazinyl group, a thianthrenyl group, a cyclopentyl group, a cyclohexyl group, an oxiranyl group, a pyrrolidinyl group, a pyrazolidinyl group, an imidazolidinyl group, a piperidinyl group, a piperazinyl group, a morpholinyl group, a di(C₆-C₅₀ aryl)amino group, a di(C₆-C₅₀ aryl)aminophenyl group, a tri(C₆-C₅₀ aryl)silyl group and derivatives thereof.

13. The organic light emitting compound of claim 1, wherein the organic light emitting compound is represented by one of Formulae 3 through 18:

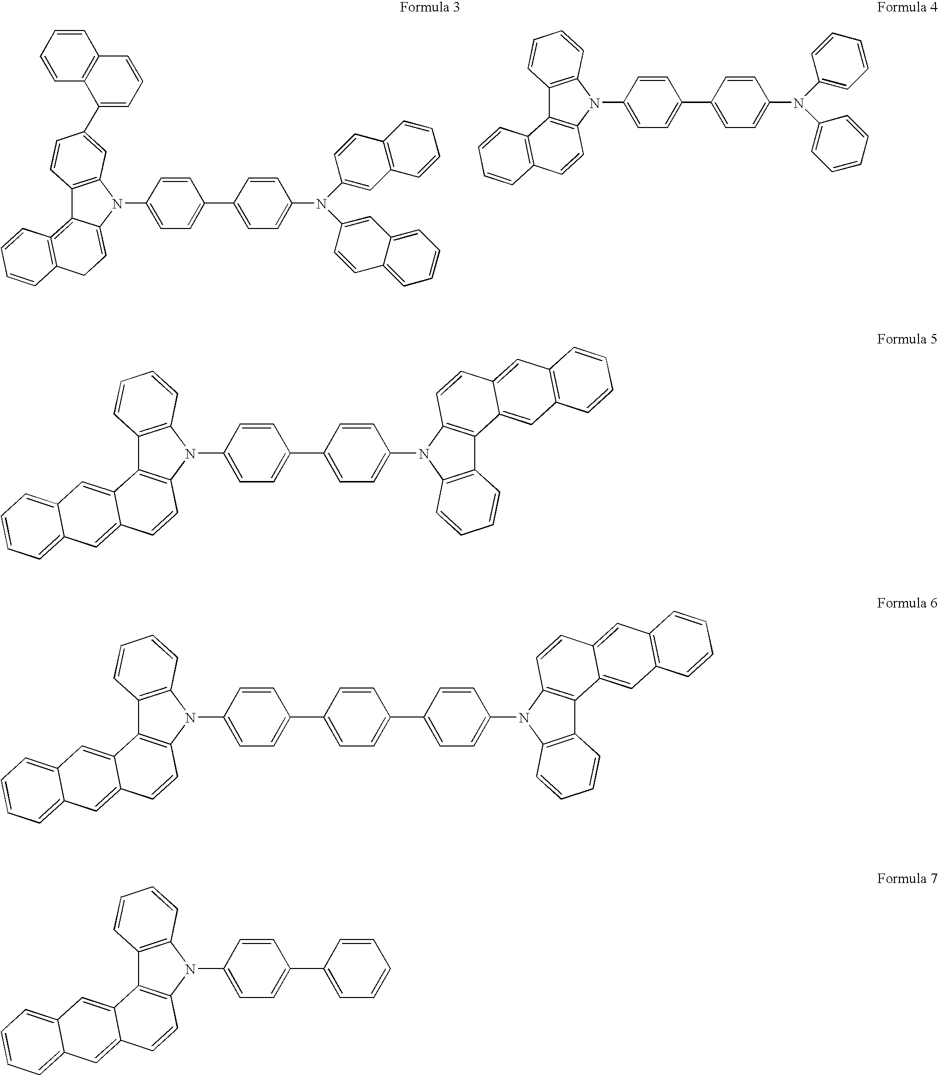

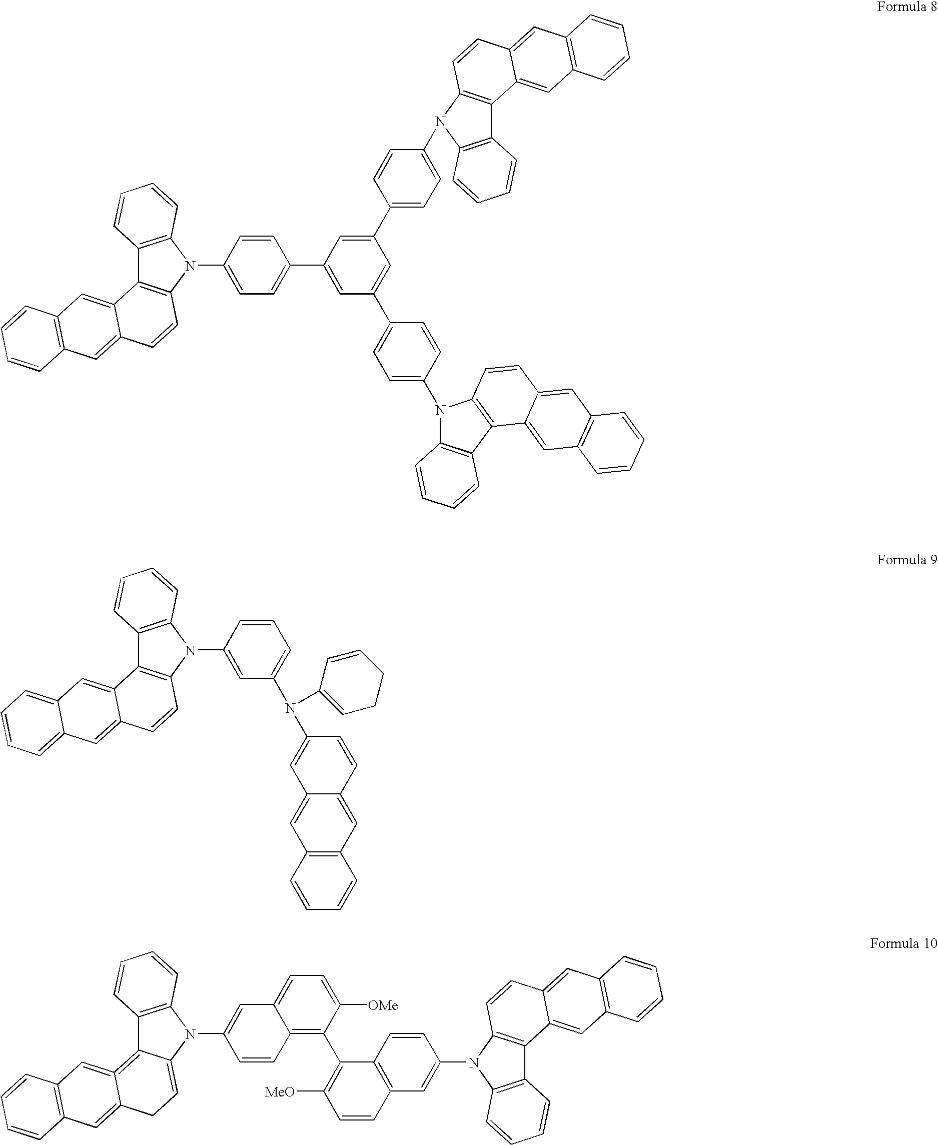

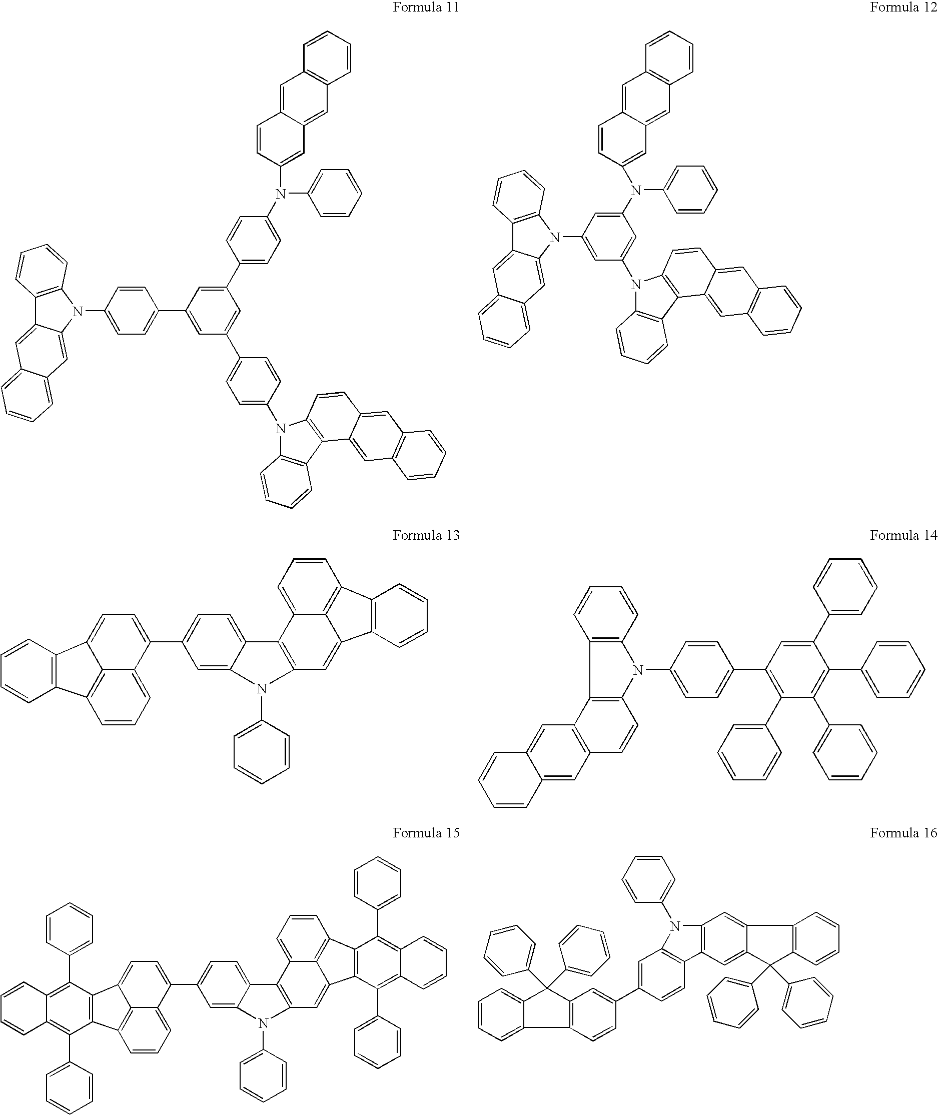

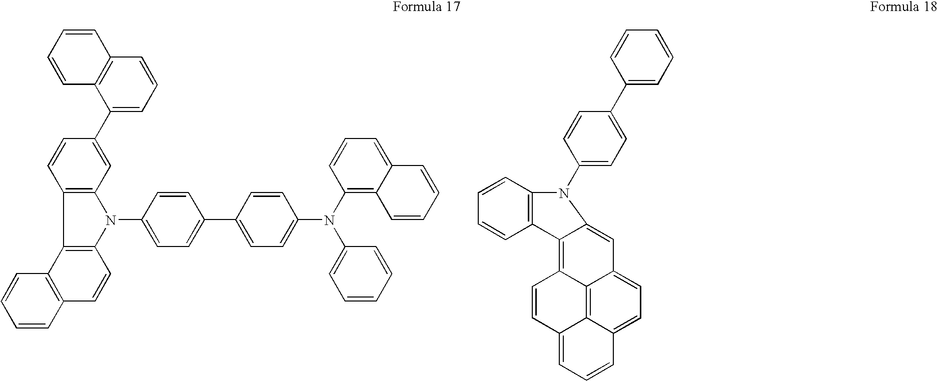

14. An organic light emitting device comprising: a first electrode; a second electrode; and an organic layer interposed between the first electrode and the second electrode, wherein the organic layer comprises an organic light emitting compound according to claim 1.

15. The organic light emitting device of claim 14, wherein the organic layer is an emission layer, a hole injection layer, or a hole transport layer.

16. The organic light emitting device of claim 14, further comprising at least one selected from the group consisting of a hole injection layer, a hole transport layer, an electron blocking layer, a hole blocking layer, an electron transport layer and an electron injection layer between the first electrode and the second electrode.

17. The organic light emitting device of claim 15, wherein the emission layer further comprises a red, green, blue or white phosphorescent or fluorescent dopant.

18. The organic light emitting device of claim 17, wherein the phosphorescent dopant contains at least one selected from the group consisting of Ir, Pt, Os, Ti, Zr, Hf, Eu, Tb, and Tm.

19. The organic light emitting device of claim 14, comprising a layered structure having a first electrode/hole injection layer/emission layer/electron transport layer/electron injection layer/second electrode structure, a first electrode/hole injection layer/hole transport layer/emission layer/electron transport layer/electron injection layer/second electrode structure or a first electrode/hole injection layer/hole transport layer/emission layer/hole blocking layer/electron transport layer/electron injection layer/second electrode structure.

20. A method of manufacturing an organic light emitting device, comprising: forming a first electrode; forming an organic thin film comprising an organic light emitting compound according to claim 1 on a surface of the first electrode; and forming a second electrode on a surface of the organic thin film opposite the first electrode.

21. The method of claim 20, wherein the organic thin film is formed using a wet spinning method or a heat transfer method.

22. The method of claim 21, wherein the wet spinning method is one selected from the group consisting of solution deposition, spin coating, inkjet printing and spray printing.
